(19) United States
(12) Patent Application Publication
(10) Pub. No.: US 2022/0276148 A1
(43) Pub. Date: Sep. 1, 2022

(54) INFORMATION PROCESSING DEVICE, DISPLAY METHOD, PROGRAM, AND INFORMATION PROCESSING SYSTEM

(71) Applicant: Sony Group Corporation, Tokyo (JP)

(72) Inventor: Kenji Yamane, Kanagawa (JP)

(73) Assignee: Sony Group Corporation, Tokyo (JP)

(21) Appl. No.: 17/635,997

(22) PCT Filed: Aug. 6, 2020
(86) PCT No.: PCT/JP2020/030151

§ 371 Date: Feb. 16, 2022

(30) Foreign Application Priority Data
Aug. 23, 2019 (JP) 2019-152592

(51) Int. Cl. G01N 15/14

(52) U.S. Cl. CPC G01N 15/1429; G01N 15/1456; G06F 3/14

(57) ABSTRACT

An information processing device includes a display control unit that displays a correspondence relationship between a gate obtained by gate analysis of an analysis target having a plurality of attributes based on the plurality of attributes and a cluster obtained by cluster analysis of the analysis target based on the plurality of attributes.

### FIELD

The present disclosure relates to an information processing device, a display method, a program, and an information processing system.

### BACKGROUND

There is an information processing device that analyzes an analysis target having a plurality of attributes on the basis of the plurality of attributes and displays an analysis result. For example, a flow cytometer analyzes a cell on the basis of a plurality of biomarkers, and displays an analysis result. In the information processing device, when the number of attributes increases, analysis is difficult. For example, in a case where two-dimensional display is performed focusing on two attributes out of N attributes, the number of combinations of the two attributes is N (N−1)/2, and when N increases, an explosion of combinations occurs. Therefore, in order to facilitate analysis, clustering and dimensional compression using machine learning are performed.

Note that Patent Literature 1 below discloses a technique of performing machine learning on detection data detected from fine particles and whether or not the fine particles are to be fractionated to create dictionary data, and determining whether or not the fine particles are to be fractionated using the dictionary data when the detection data is supplied. According to this technique, time required for determining whether or not the fine particles are to be fractionated can be shortened, and the fine particles can be fractionated on the basis of a determination result.

### CITATION LIST

### Patent Literature

Patent Literature 1: WO 2018/198586 A

### SUMMARY

### Technical Problem

Since a result of analysis performed while two attributes of interest are changed and a result of analysis obtained by clustering or dimensional compression are displayed separately, it is difficult for a user to cause the two analysis results to correspond to each other.

In view of the above circumstances, an object of the present technology is to provide an information processing device, a display method, a program, and an information processing system that support correspondence between two analysis results.

### Solution to Problem

To solve the above problem, an information processing device according to the present disclosure includes a display control unit that displays a correspondence relationship between a gate obtained by gate analysis of an analysis target having a plurality of attributes based on the plurality of attributes and a cluster obtained by cluster analysis of the analysis target based on the plurality of attributes.

### BRIEF DESCRIPTION OF DRAWINGS

FIG. 1 is a block diagram illustrating a configuration of an information processing system according to a first embodiment.

FIG. 2 is a diagram illustrating an example of a gate information storage unit.

FIG. 3 is a diagram illustrating an example of a clustering data storage unit.

FIG. 4A is a first diagram illustrating a calculation example of a matching degree using a confusion matrix.

FIG. 4B is a second diagram illustrating a calculation example of a matching degree using a confusion matrix.

FIG. 4C is a third diagram illustrating a calculation example of a matching degree using a confusion matrix.

FIG. 5 is a diagram illustrating an analysis example.

FIG. 6 is a flowchart illustrating a flow of processing performed by a gate processing unit.

FIG. 7 is a flowchart illustrating a flow of processing performed by a clustering processing unit.

FIG. 8 is a flowchart illustrating a flow of processing performed by a matching degree calculation unit.

FIG. 9 is a block diagram illustrating a configuration of an information processing system according to a second embodiment.

FIG. 10 is a flowchart illustrating a flow of processing performed by an information processing device.

FIG. 11 is a diagram illustrating a display example.

FIG. 12 is a block diagram illustrating a hardware configuration example of an information processing device according to an embodiment of the present disclosure.

### DESCRIPTION OF EMBODIMENTS

Hereinafter, an embodiment of the present disclosure will be described in detail with reference to the drawings. Note that in the following embodiments, the same portion is denoted by the same reference numeral, and redundant description will be omitted.

In addition, the present disclosure will be described according to the following item order.

1. First embodiment

1.1 Configuration of information processing system

1.2 Method for calculating matching degree

1.3 Analysis example

1.4 Analysis operation

1.5 Action and effect

2. Second embodiment

2.1 Configuration of information processing system

2.2 Analysis operation

2.3 Display example

2.4 Action and effect

3. Hardware configuration of information processing device

### 1. First Embodiment

Hereinafter, an information processing device, a display method, a program, and an information processing system according to a first embodiment of the present disclosure will be described in detail with reference to the drawings.

1.1 Configuration of Information Processing System

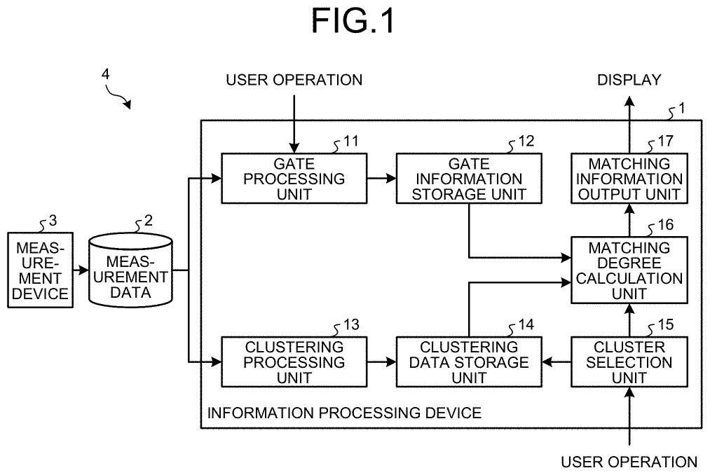

First, a configuration of the information processing system will be described. FIG. 1 is a block diagram illustrating the configuration of the information processing system according to the first embodiment. As illustrated in FIG. 1, an information processing system 4 includes an information processing device 1 and a measurement device 3.

The measurement device 3 is a measurement device capable of detecting fluorescence of each color from a cell or the like as a measurement sample. The measurement device 3 is, for example, a flow cytometer that detects fluorescence of each color from a cell by causing a fluorescently stained cell to flow through a flow cell at a high speed and irradiating the flowing cell with a light beam. A measurement sample measured with the flow cytometer may be a biologically derived particle such as a microorganism or a biologically relevant particle in addition to the cell. For example, the cell may be an animal cell (for example, a corpuscle-based cell) or a plant cell. For example, the microorganism may be a bacterium such as Escherichia coli, a virus such as tobacco mosaic virus, or a fungus such as yeast. The biologically relevant particle may be a particle constituting a cell such as a chromosome, a liposome, mitochondria, or various organelles. Note that the biologically relevant particle may include a biologically relevant polymer such as a nucleic acid, a protein, a lipid, a sugar chain, or a complex thereof. Each of these biologically derived particles may have either a spherical shape or a non-spherical shape, and is not particularly limited in size and mass.

In addition, the measurement sample may be an industrially synthesized particle such as a latex particle, a gel particle, or an industrial particle. For example, the industrially synthesized particle may be a particle synthesized with an organic resin material such as polystyrene or polymethyl methacrylate, an inorganic material such as glass, silica, or a magnetic body, or a metal such as gold colloid or aluminum. Similarly, each of these industrially synthesized particles may have either a spherical shape or a non-spherical shape, and is not particularly limited in size and mass.

The measurement sample can be labeled (stained) with one or more fluorescent dyes prior to measurement of a fluorescence spectrum. The measurement sample may be labeled with a fluorescent dye by a known method. Specifically, when the measurement sample is a cell, a fluorescently labeled antibody that is selectively bonded to an antigen present on a surface of a cell is mixed with a cell to be measured, and the fluorescently labeled antibody is bonded to an antigen on a surface of the cell. As a result, the cell to be measured can be labeled with a fluorescent dye. Alternatively, the cell to be measured can be labeled with a fluorescent dye by mixing a fluorescent dye that is selectively taken into a specific cell with the cell to be measured.

The fluorescently labeled antibody is an antibody to which a fluorescent dye is bonded as a label. The fluorescently labeled antibody may be an antibody to which a fluorescent dye is directly bonded. Alternatively, the fluorescently labeled antibody may be an antibody obtained by bonding a fluorescent dye to which avidin is bonded to a biotin-labeled antibody by an avidin-biotin reaction. Note that as the antibody, either a polyclonal antibody or a monoclonal antibody can be used.

The fluorescent dye for labeling a cell is not particularly limited, and at least one or more known dyes used for staining a cell or the like can be used. For example, as the fluorescent dye, phycoerythrin (PE), fluorescein isothiocyanate (FITC), PE-Cy5, PE-Cy7, PE-Texas Red (registered trademark), allophycocyanin (APC), APC-Cy7, ethidium bromide, propidium iodide, Hoechst (registered trademark) 33258, Hoechst (registered trademark) 33342, 4′,6-diamidino-2-phenylindole (DAPI), acridineorange, chromomycin, mithramycin, olivomycin, pyronin Y, thiazole orange, rhodamine 101, isothiocyanate, BCECF, BCECF-AM, C. SNARF-1, C. SNARF-1-AMA, aequorin, Indo-1, Indo-1-AM, Fluo-3, Fluo-3-AM, Fura-2, Fura-2-AM, oxonol, Texas Red (registered trademark), Rhodamine 123, 10-N-nony-acridine orange, fluorescein, fluorescein diacetate, carboxyfluorescein, carboxyfluorescein diacetate, carboxydichlorofluorescein, and carboxydichlorofluorescein diacetate can be used. In addition, derivatives of the above-described fluorescent dyes and the like can also be used.

The flow cytometer includes a laser light source that emits laser light having a wavelength capable of exciting a fluorescent dye with which a measurement sample is labeled, a flow cell through which the measurement sample flows in one direction, and a photodetector that receives fluorescence, phosphorescence, or scattered light from the measurement sample irradiated with the laser light.

The laser light source is, for example, a semiconductor laser light source that emits laser light having a predetermined wavelength. A plurality of laser light sources may be disposed. When the plurality of laser light sources is disposed, positions irradiated with laser light from the laser light sources may be the same as or different from each other in the flow cell. However, in a case where different positions are irradiated with laser light from the plurality of laser light sources, light from the measurement sample can be detected by different photodetectors. Therefore, even in a case where dyes that emit light beams having close wavelengths are used, measurement can be performed without color mixing. Note that the laser light emitted from the laser light source may be either pulsed light or continuous light. For example, as the laser light source, a plurality of semiconductor laser light sources that emits laser light having a wavelength of 480 nm and laser light having a wavelength of 640 nm may be used.

The flow cell is a flow path through which a plurality of measurement samples flows in line in one direction. Specifically, through the flow cell, a sheath liquid enclosing the measurement samples flows at a high speed as a laminar flow, and the plurality of measurement samples thereby flows in line in one direction. The flow cell can be formed in a microchip or a cuvette.

The photodetector detects fluorescence, phosphorescence, and scattered light generated from a measurement sample irradiated with laser light by photoelectric conversion.

For example, the photodetector may include a detector that detects scattered light LS including forward scattered light and side scattered light from a measurement sample, and a light receiving element array that detects fluorescence from the measurement sample.

The detector may be, for example, a known photoelectric conversion element such as a charge coupled device (CCD), a complementary metal oxide semiconductor (CMOS), or a photodiode. The light receiving element array can be constituted by, for example, arranging a plurality of independent detection channels having different wavelength ranges of light to be detected. Specifically, the light receiving element array may be, for example, a light receiving element array in which a plurality of photo multiplier tubes (PMTs) or photodiodes having different wavelength ranges to be detected is arranged one-dimensionally or the like. The light receiving element array photoelectrically converts fluorescence of a measurement sample dispersed into a spectrum by a spectroscopic element such as a prism or a grating.

As a result, in the flow cytometer, first, each measurement sample flowing through the flow cell is irradiated with laser light emitted from the laser light source. The measurement sample emits scattered light and fluorescence (or phosphorescence) by being irradiated with the laser light. Here, the scattered light emitted from the measurement sample is detected by the detector. Meanwhile, the fluorescence emitted from the measurement sample is detected by being dispersed into a continuous spectrum by the spectroscopic element and then received by the light receiving element array.

The measurement device 3 may be a biological microscope such as a fluorescence microscope or a confocal laser microscope that fluorescently observes an observation sample such as a cell or a tissue labeled or stained with a fluorescent dye to detect fluorescence of each color from the observation sample. The observation sample may be, for example, a pathological sample such as a tissue, a cell, or blood collected from a patient, a biological sample such as a cultured cell, a fertilized egg, or a sperm, or a biological material such as a cell sheet or a three-dimensional cell tissue. The biological microscope may acquire not only light information such as fluorescence, phosphorescence, or scattered light from the observation sample but also image information such as form information such as the length or size of the observation sample or position information.

The measurement device 3 outputs a detection result as measurement data 2. The measurement data 2 includes an intensity value for each wavelength region for each cell. The measurement device 3 transfers the measurement data 2 to, for example, the information processing device 1.

The information processing device 1 acquires the measurement data 2 measured by the measurement device 3 and calculates a fluorescence intensity corresponding to each fluorescent dye. Then, the information processing device 1 analyzes a cell on the basis of the calculated fluorescence intensity, and displays an analysis result. Here, the cell is an example of an analysis target, and the analysis target may be a measurement sample of the flow cytometer or an observation sample of the biological microscope. In addition, the fluorescence intensity is an example of an attribute, and the attribute only needs to be an attribute indicated by the measurement sample or the observation sample, and may be light information such as fluorescence or scattered light or image information such as form information or position information. Note that the information processing device 1 and the measurement device 3 may be connected to each other via a network, and the information processing device 1 may acquire the measurement data 2 via the network.

The information processing device 1 includes a gate processing unit 11, a gate information storage unit 12, a clustering processing unit 13, a clustering data storage unit 14, a cluster selection unit 15, a matching degree calculation unit 16, and a matching information output unit 17. Note that all or some of these functional units may be performed in a cloud. For example, the clustering processing unit 13, the clustering data storage unit 14, the cluster selection unit 15, and the matching degree calculation unit 16 may be performed in a cloud. In this case, the measurement data 2 is also transferred to the cloud.

The gate processing unit 11 reads the measurement data 2, performs gating on the basis of user operation, and stores a gating result in the gate information storage unit 12. The gate processing unit 11 reads the measurement data 2 from a file, for example. The gate processing unit 11 receives, for example, user operation using a touch panel, a mouse, or a keyboard.

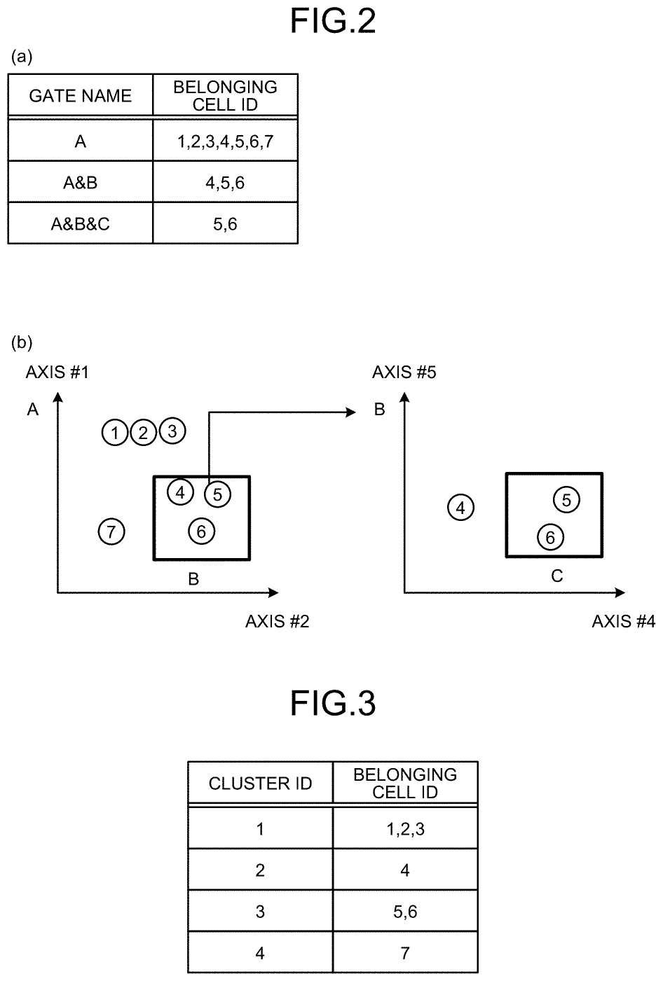

The gate information storage unit 12 stores a gating result obtained by the gate processing unit 11. FIG. 2 is a diagram illustrating an example of the gate information storage unit 12. FIG. 2(a) illustrates the gate information storage unit 12, and FIG. 2(b) illustrates a gate.

In FIG. 2(b), a number surrounded by a circle indicates a cell ID for identifying a cell. As illustrated in FIG. 2(b), seven cells of cell #1 to cell #7 belong to gate A. Gate B is a gate created from gate A on the basis of axis #1 and axis #2. Three cells of cell #4, cell #5, and cell #6 belong to gate B. Gate C is a gate created on the basis of axis #4 and axis #5 from gate B. Two cells of cell #5 and cell #6 belong to gate C.

As illustrated in FIG. 2(a), the gate information storage unit 12 stores a gate name and a belonging cell ID while the gate name and the belonging cell ID correspond to each other for each gate. The gate name is a name for identifying a gate. The belonging cell ID is a cell ID of a cell belonging to a gate.

For example, seven cells of cell #1 to cell #7 belong to gate A, three cells of cell #4, cell #5, and cell #6 belong to gate A & B, and two cells of cell #5 and cell #6 belong to gate A & B & C. Note that gate A & B indicates that gate B is created from gate A.

The clustering processing unit 13 reads the measurement data 2, performs clustering, and stores a clustering result in the clustering data storage unit 14. K is designated, for example, as K-means, and the clustering processing unit 13 classifies the measurement data 2 into K clusters. Alternatively, the clustering processing unit 13 may automatically determine the number of divisions as in Flow self-organizing map (FlowSOM).

Alternatively, for example, as in T-SNE, the clustering processing unit 13 may perform dimensional compression and perform gating on a result of the dimension compression to perform clustering. Alternatively, the clustering processing unit 13 may perform two-stage clustering such as meta-clustering and use two cluster definitions such as a cluster ID and a meta-cluster ID. Here, the meta-cluster is a collection of clusters.

The clustering data storage unit 14 stores a clustering result obtained by the clustering processing unit 13. FIG. 3 is a diagram illustrating an example of the clustering data storage unit 14. As illustrated in FIG. 3, the clustering data storage unit 14 stores a cluster ID and a belonging cell ID while the cluster ID and the belonging cell ID correspond to each other for each cluster. The cluster ID is a number for identifying a cluster. The belonging cell ID is a cell ID of a cell belonging to a cluster.

For example, three cells of cell #1, cell #2, and cell# 3 belong to cluster #1, cell #4 belongs to cluster #2, cell# 5 and cell# 6 belong to cluster #3, and cell #7 belongs to cluster #4. Note that the clustering data storage unit 14 may further store a meta-cluster ID.

The cluster selection unit 15 selects a cluster on the basis of user operation, and notifies the matching degree calculation unit 16 of the cluster ID of the selected cluster. For example, the cluster selection unit 15 receives user operation using a touch panel, a mouse, or a keyboard.

The matching degree calculation unit 16 calculates a matching degree between a cluster whose cluster ID the matching degree calculation unit 16 has been notified of by the cluster selection unit 15 and a gate for all the gates using a confusion matrix, and notifies the matching information output unit 17 of the name of a gate having the highest matching degree.

The matching degree calculation unit 16 may notify the matching information output unit 17 of the name of a gate in descending order of the matching degree instead of notifying the matching information output unit 17 of the name of a gate having the highest matching degree. In addition, the matching degree calculation unit 16 may notify the matching information output unit 17 of the matching degree together with the name of a gate.

The matching information output unit 17 highlights a gate whose name the matching information output unit 17 has been notified of by the matching degree calculation unit 16 on a display device. Examples of a highlighting method include displaying the gate in a color different from the other gates, changing a line constituting the gate by blinking or thickening, changing the color, shape, or the like of a plot in the gate, and changing a background color in the gate. In addition, the matching information output unit 17 may use, as a highlighting color, the same color or the same type color (a similar color, a color with a different color tone, or the like) as the color of a cluster displayed on a clustering side. In addition, the matching information output unit 17 may highlight a parent gate in addition to the gate whose name the matching information output unit 17 has been notified of by the matching degree calculation unit 16. In a case where the matching information output unit 17 is notified of a gate name in descending order of the matching degree, for example, the matching information output unit 17 may display a gate by changing the color of the gate in order of the matching degree. In addition, the matching information output unit 17 may display the matching degree in a gate.

1.2 Method for Calculating Matching Degree

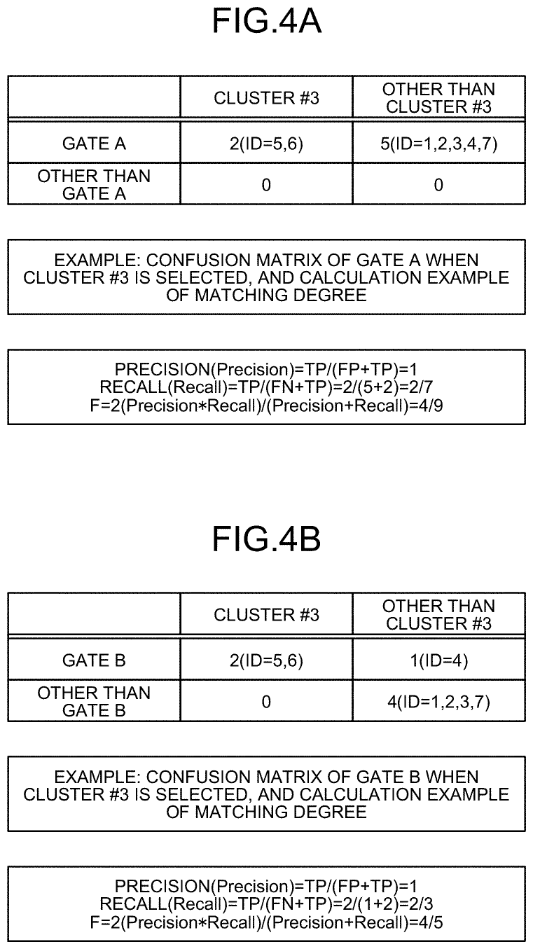

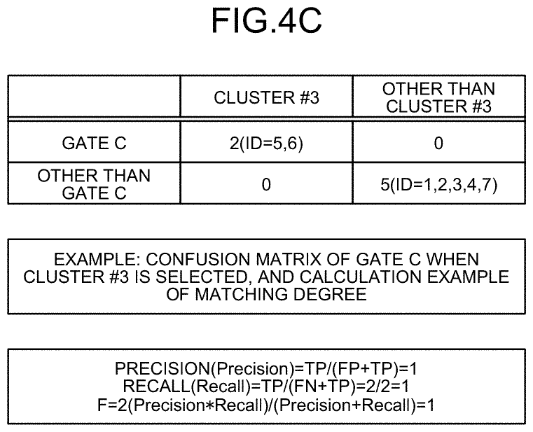

Next, an example of a method for calculating a matching degree will be described. FIGS. 4A to 4C are diagrams illustrating calculation examples of a matching degree using a confusion matrix. FIG. 4A illustrates a calculation example of a matching degree between gate A and cluster #3. FIG. 4B illustrates a calculation example of a matching degree between gate B and cluster #3. FIG. 4C illustrates a calculation example of a matching degree between gate C and cluster #3.

In FIGS. 4A to 4C, the confusion matrix is a matrix in which the number of cells belonging to a gate for which a matching degree is calculated and the number of cells belonging to a gate other than the gate for which the matching degree is calculated are set as rows. In addition, the confusion matrix is a matrix in which the number of cells belonging to a cluster for which the matching degree is calculated and the number of cells belonging to a cluster other than the cluster for which the matching degree is calculated are set as columns.

In the confusion matrix, the number of cells belonging to the gate for which the matching degree is calculated and belonging to the cluster for which the matching degree is calculated is represented by True Positive (TP). In addition, the number of cells belonging to the gate for which the matching degree is calculated and belonging to a cluster other than the cluster for which the matching degree is calculated is represented by False Negative (FN). In addition, the number of cells belonging to a gate other than the gate for which the matching degree is calculated and belonging to the cluster for which the matching degree is calculated is represented by False Positive (FP). In addition, the number of cells belonging to a gate other than the gate for which the matching degree is calculated and belonging to a cluster other than the cluster for which the matching degree is calculated is represented by True Negative (TN).

Then, assuming that precision=TP/(FP+TP) and recall=TP/(FN+TP), the matching degree calculation unit 16 calculates a matching degree F. by using F=2 (precision recall)/(precision+recall).

In FIG. 4A, in order to calculate a matching degree between gate A and cluster #3, the confusion matrix is a matrix in which the number of cells belonging to gate A and the number of cells belonging to a gate other than gate A are set as rows, and the number of cells belonging to cluster #3 and the number of cells belonging to a cluster other than cluster #3 are set as columns.

As illustrated in FIG. 4A, since cell #5 and cell #6 belong to gate A and belong to cluster #3, TP=2. Since cell #1, cell #2, cell #3, cell #4, and cell #7 belong to gate A and belong to a cluster other than cluster #3, FN=5. Since there is no cell belonging to a gate other than gate A, FP=TN=0.

Therefore, precision=TP/(FP+TP)=2/(0+2)=1, and recall=TP/(FN+TP)=2/(5+2)=2/7. In addition, matching degree=2 (precision recall)/(precision+recall)=2*1*(2/7)/(1+2/7)=(4/7)/(9/7)=4/9.

In FIG. 4B, in order to calculate a matching degree between gate B and cluster #3, the confusion matrix is a matrix in which the number of cells belonging to gate B and the number of cells belonging to a gate other than gate B are set as rows, and the number of cells belonging to cluster #3 and the number of cells belonging to a cluster other than cluster #3 are set as columns.

As illustrated in FIG. 4B, since cell #5 and cell #6 belong to gate B and belong to cluster #3, TP=2. Since cell #4 belongs to gate B and belongs to a cluster other than cluster #3, FN=1. Since there is no cell belonging to a gate other than gate B and belonging to cluster #3, FP=0. Since cell #1, cell #2, cell #3, and cell #7 belong to a gate other than gate B and belong to a cluster other than cluster #3, TN=4.

Therefore, precision=TP/(FP+TP)=2/(0+2)=1, and recall=TP/(FN+TP)=2/(1+2)=2/3. In addition, matching degree=2 (precision recall)/(precision+recall)=2*1*(2/3)/(1+2/3)=(4/3)/(5/3)=4/5.

In FIG. 4C, in order to calculate a matching degree between gate C and cluster #3, the confusion matrix is a matrix in which the number of cells belonging to gate C and the number of cells belonging to a gate other than gate C are set as rows, and the number of cells belonging to cluster #3 and the number of cells belonging to a cluster other than cluster #3 are set as columns.

As illustrated in FIG. 4C, since cell #5 and cell #6 belong to gate C and belong to cluster #3, TP=2. Since there is no cell belonging to gate C and belonging to a cluster other than cluster #3, FN=0. Since there is no cell belonging to a gate other than gate C and belonging to cluster #3, FP=0. Since cell #1, cell #2, cell #3, cell #4, and cell #7 belong to a gate other than gate C and belong to a cluster other than cluster #3, TN=5.

Therefore, precision=TP/(FP+TP)=2/(0+2)=1, and recall=TP/(FN+TP)=2/(0+2)=2/2=1. In addition, matching degree=2 (precision*recall)/(precision+recall)=2*1*1/(1+1)=2/2=1.

1.3 Analysis Example

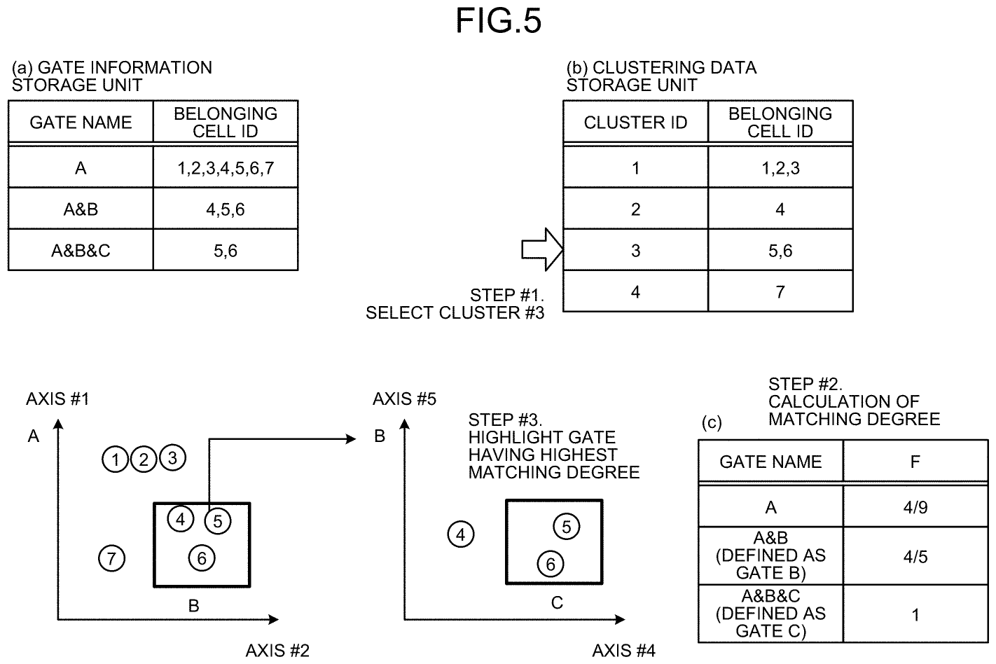

Next, an analysis example will be described. FIG. 5 is a diagram illustrating an analysis example. FIG. 5(a) illustrates an example of the gate information storage unit 12, FIG. 5(b) illustrates an example of the clustering data storage unit 14, and FIG. 5(c) illustrates a matching degree.

As step #1, cluster #3 is selected by a user. Then, as step #2, a matching degree with cluster #3 is calculated for all the gates. As illustrated in FIG. 5(c), the matching degree of gate A is 4/9, the matching degree of gate B is 4/5, and the matching degree of gate C is 1.

Therefore, since the matching degree of gate C is the highest, gate C is highlighted. In FIG. 5, gate C is displayed in a thick frame, but in an actual screen, for example, gate C is displayed in a red thick frame. In addition, gate B that is a parent of gate C may also be highlighted. In FIG. 5, gate B is also displayed in a thick frame, but in an actual screen, for example, gate B is displayed in a blue thick frame having a color different from gate C.

1.4 Analysis Operation

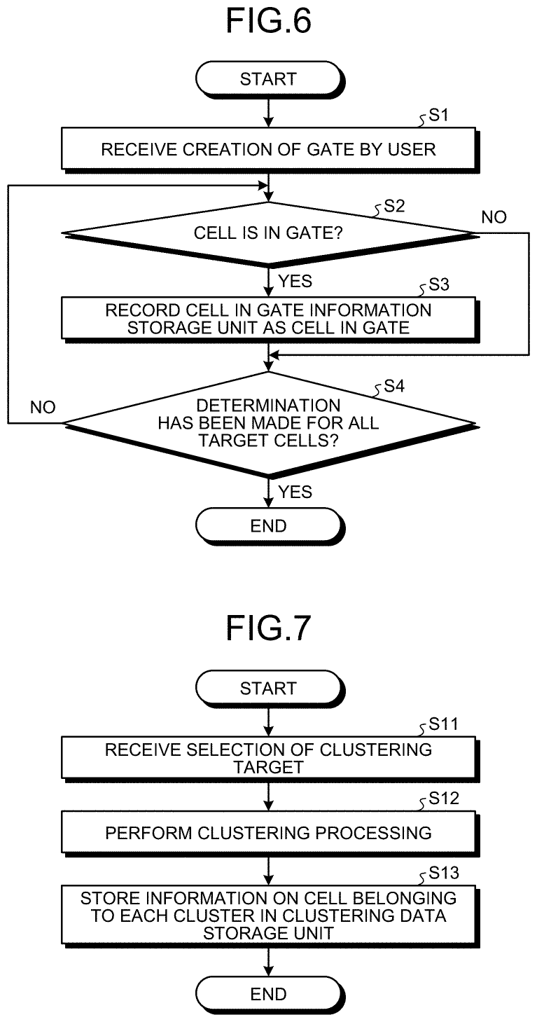

Next, analysis operation performed by the information processing device 1 will be described with reference to FIGS. 6 to 8. FIG. 6 is a flowchart illustrating a flow of processing performed by the gate processing unit 11. As illustrated in FIG. 6, the gate processing unit 11 receives creation of a gate by a user (step S1).

Then, the gate processing unit 11 determines whether or not one cell among target cells is in the gate (step S2). If the cell is in the gate, the gate processing unit 11 records the cell in the gate information storage unit 12 as a cell in the gate (step S3).

Then, the gate processing unit 11 determines whether or not the determination as to whether or not a target cell is in the gate has been made for all the target cells (step S4). If there is a target cell for which the determination as to whether or not the target cell is in the gate has not been made, the process returns to step S2. Meanwhile, if the determination as to whether or not a target cell is in the gate has been made for all the target cells, the gate processing unit 11 ends the processing.

As described above, since the gate processing unit 11 records information on a cell belonging to the gate in the gate information storage unit 12, the matching degree calculation unit 16 can calculate a matching degree of each gate using the information stored in the gate information storage unit 12.

FIG. 7 is a flowchart illustrating a flow of processing performed by the clustering processing unit 13. As illustrated in FIG. 7, the clustering processing unit 13 receives selection of a clustering target from a user (step S11), and performs clustering processing on the selected clustering target (step S12). Then, the clustering processing unit 13 stores information on a cell belonging to each cluster in the clustering data storage unit 14 (step S13).

As described above, since the clustering processing unit 13 stores the information on a cell belonging to each cluster in the clustering data storage unit 14, the matching degree calculation unit 16 can calculate a matching degree of each gate using the information stored in the clustering data storage unit 14.

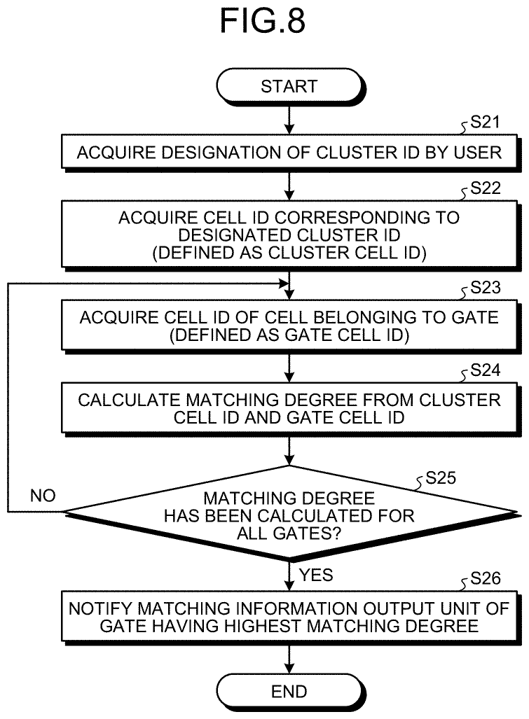

FIG. 8 is a flowchart illustrating a flow of processing performed by the matching degree calculation unit 16. As illustrated in FIG. 8, the matching degree calculation unit 16 acquires designation of a cluster ID by a user from the cluster selection unit 15 (step S21), and acquires a cell ID corresponding to the designated cluster ID (step S22). Here, the acquired cell ID is defined as a cluster cell ID.

Then, the matching degree calculation unit 16 acquires a cell ID of a cell belonging to one gate (step S23). Here, the acquired cell ID is defined as a gate cell ID. Then, the matching degree calculation unit 16 calculates a matching degree from the cluster cell ID and the gate cell ID (step S24).

Then, the matching degree calculation unit 16 determines whether or not the matching degree has been calculated for all the gates (step S25). If there is a gate for which the matching degree has not been calculated, the process returns to step S23. Meanwhile, if the matching degree has been calculated for all the gates, the matching degree calculation unit 16 notifies the matching information output unit 17 of a gate having the highest matching degree (step S26).

As described above, since the matching degree calculation unit 16 notifies the matching information output unit 17 of a gate having the highest matching degree, the matching information output unit 17 can highlight the gate having the highest matching degree.

1.5 Action and Effect

As described above, according to the first embodiment, the matching degree calculation unit 16 calculates a matching degree between a cluster designated by a user and a gate for all the gates, and notifies the matching information output unit 17 of a gate having the highest matching degree. Then, the matching information output unit 17 highlights the gate having the highest matching degree.

Therefore, the information processing device 1 can support correspondence between a result of gate analysis and a result of cluster analysis. Therefore, for example, a user can specify whether or not clustering has succeeded.

In addition, the information processing device 1 can visualize in which gate a clustered population is located in normal gate analysis. In this case, since a gate in normal gate analysis represents the phenotype of a cell (type of a cell), by such visualization, it is possible to visualize correspondence information on which biological population the clustered population represents, and it is possible to encourage a user to make a new discovery in analysis.

In addition, in the first embodiment, since the matching degree calculation unit 16 calculates a matching degree using a confusion matrix based on the number of cells included in a gate and the number of cells included in a cluster, it is possible to accurately calculate the matching degree.

Note that in the first embodiment, designation of a cluster is received from a user, but the information processing device 1 may receive designation of a gate from the user, and may highlight a cluster having the highest matching degree with the received gate. In addition, in the first embodiment, a matching degree is calculated using a confusion matrix based on the number of cells included in a gate and the number of cells included in a cluster, but the information processing device 1 may calculate the matching degree by another method. In addition, in the first embodiment, a cluster and a gate are caused to correspond to each other on the basis of a matching degree, but the information processing devices 1 and 1a may cause a cluster and a gate to correspond to each other on the basis of another value or another correspondence relationship.

In addition, in the first embodiment, the case of acquiring the measurement data 2 measured by the measurement device 3 has been described, but the information processing device 1 may acquire data having a plurality of attributes instead of the measurement data 2.

### 2. Second Embodiment

By the way, in the above first embodiment, a gate having the highest matching degree with a cluster designated by a user is highlighted. However, the information processing device 1 can also specify a cluster closest to each gate and display each gate in a color corresponding to the specified cluster. Therefore, in a second embodiment, an information processing device that specifies a cluster closest to each gate and displays each gate in a color corresponding to the specified cluster will be described.

2.1 Configuration of Information Processing System

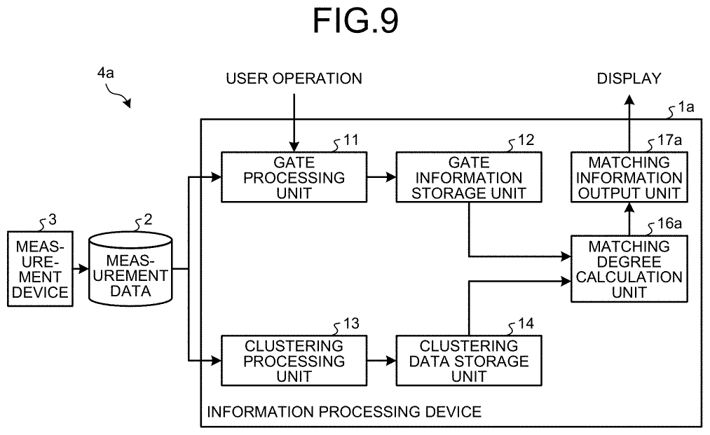

FIG. 9 is a block diagram illustrating a configuration of an information processing system according to the second embodiment. As illustrated in FIG. 9, as compared with the information processing system 4 according to the first embodiment illustrated in FIG. 1, an information processing system 4a according to the second embodiment includes an information processing device 1a instead of the information processing device 1.

As compared with the information processing device 1, the information processing device 1a includes a matching degree calculation unit 16a and a matching information output unit 17a instead of the matching degree calculation unit 16 and the matching information output unit 17, respectively, and does not include the cluster selection unit 15.

The matching degree calculation unit 16 specifies a cluster having the highest matching degree for all the gates, and notifies the matching information output unit 17a of the name of the specified cluster. The matching information output unit 17a highlights each of the gates on a display device in a color corresponding to a cluster on the basis of the name of the cluster of which the matching information output unit 17a has been notified by the matching degree calculation unit 16a.

2.2 Analysis Operation

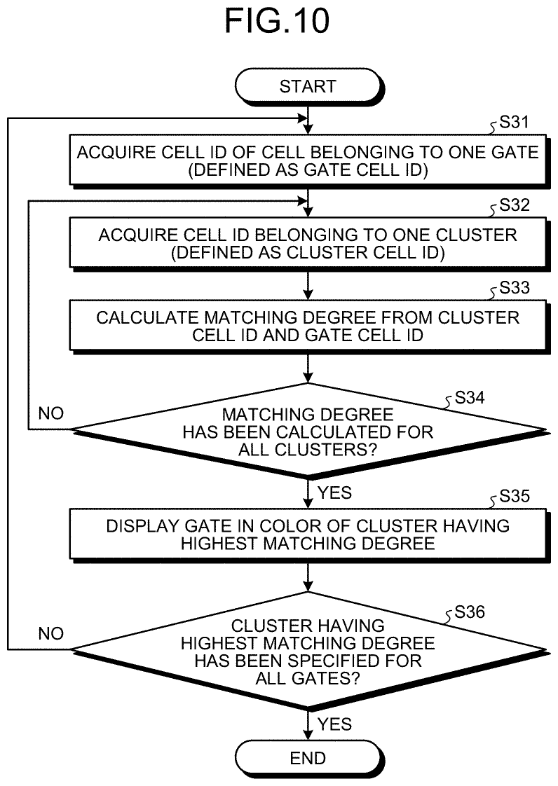

FIG. 10 is a flowchart illustrating a flow of processing performed by the information processing device 1a. Note that in FIG. 10, it is assumed that a gate and a cluster are created. As illustrated in FIG. 10, the information processing device 1a acquires a cell ID of a cell belonging to one gate (step S31), and acquires a cell ID belonging to one cluster (step S32).

Then, the information processing device 1a calculates a matching degree from the cluster cell ID and the gate cell ID (step S33). Then, the information processing device 1a determines whether or not the matching degree has been calculated for all the clusters (step S34). If there is a cluster for which the matching degree has not been calculated, the process returns to step S32.

Meanwhile, if the matching degree has been calculated for all the clusters, the information processing device 1a displays a gate in a color of a cluster having the highest matching degree (step S35). Then, the information processing device 1a determines whether or not a cluster having the highest matching degree has been specified for all the gates (step S36). If there is a gate for which a cluster having the highest matching degree has not been specified, the process returns to step S31. Meanwhile, if a cluster having the highest matching degree is specified for all the gates, the information processing device 1a ends the processing.

As described above, since the information processing device 1a displays each of the gates in a color of a cluster having the highest matching degree, it is possible to support correspondence between a cluster and a gate by a user.

2.3 Display Example

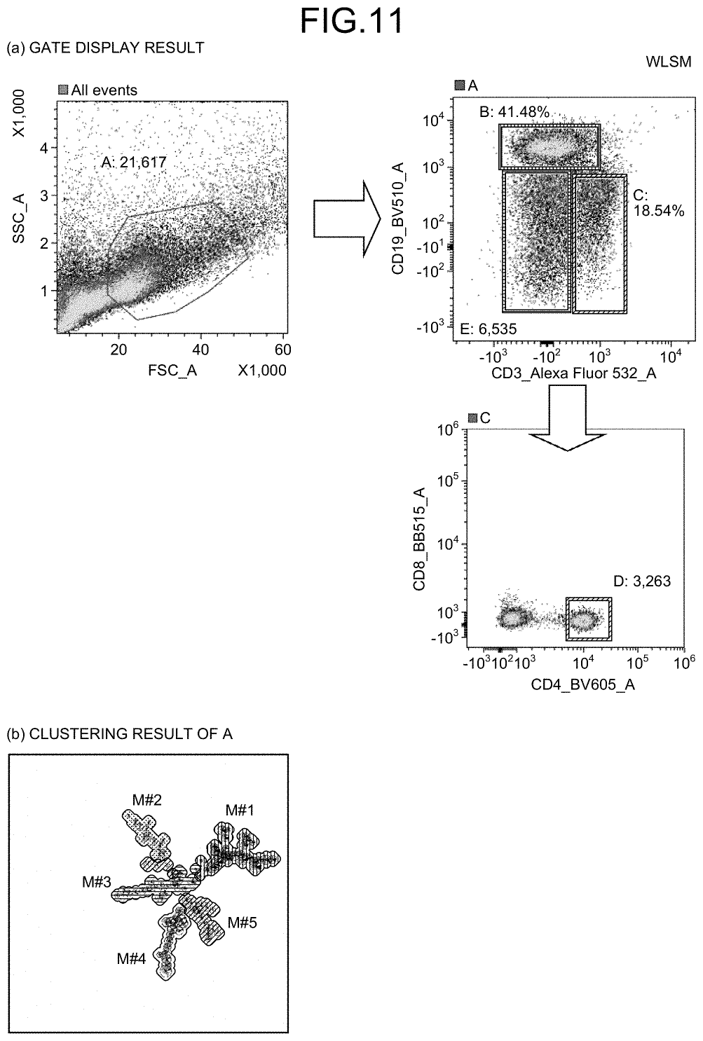

FIG. 11 is a diagram illustrating a display example. FIG. 11(a) illustrates a gate display result, and FIG. 11(b) illustrates a clustering result. In FIG. 11(a), a gate is applied at A, B gate, C gate, and E gate are applied in gate A, and D gate is applied in C gate. FIG. 11(b) illustrates a clustering result of A. In FIG. 11(b), one circle indicates one cluster. As illustrated in FIG. 11(b), A is clustered into five meta-clusters represented by M#1 to M#5. Note that, in FIG. 11(b), different types of shading (including no shading) are performed on the meta-clusters, but in an actual display device, the meta-clusters are displayed in different colors.

As illustrated in FIG. 11(a), since gate B has the highest matching degree with meta-cluster M#1, gate B is shaded in the same manner (displayed in the same color) as meta-cluster M#1. Since gate C and gate D have the highest matching degree with meta-cluster M#5, gate C and gate D are shaded in the same manner as meta-cluster M#5. Since gate E has the highest matching degree with meta-cluster M#3, gate E is shaded in the same manner as meta-cluster M#3.

2.4 Action and Effect

As described above, according to the second embodiment, the matching degree calculation unit 16a specifies a cluster having the highest matching degree for all the gates, and notifies the matching information output unit 17a of a cluster ID of the specified cluster. Then, the matching information output unit 17a displays each of the gates in a color of the cluster having the highest matching degree. Therefore, the information processing device 1a can support correspondence between a result of gate analysis and a result of cluster analysis. Therefore, for example, a user can specify whether or not clustering has succeeded. In addition, a user can usually specify the phenotype of a cell by gate analysis, and the information processing device 1a can visualize at which position the phenotype is clustered.

3. Hardware Configuration of Information Processing Device

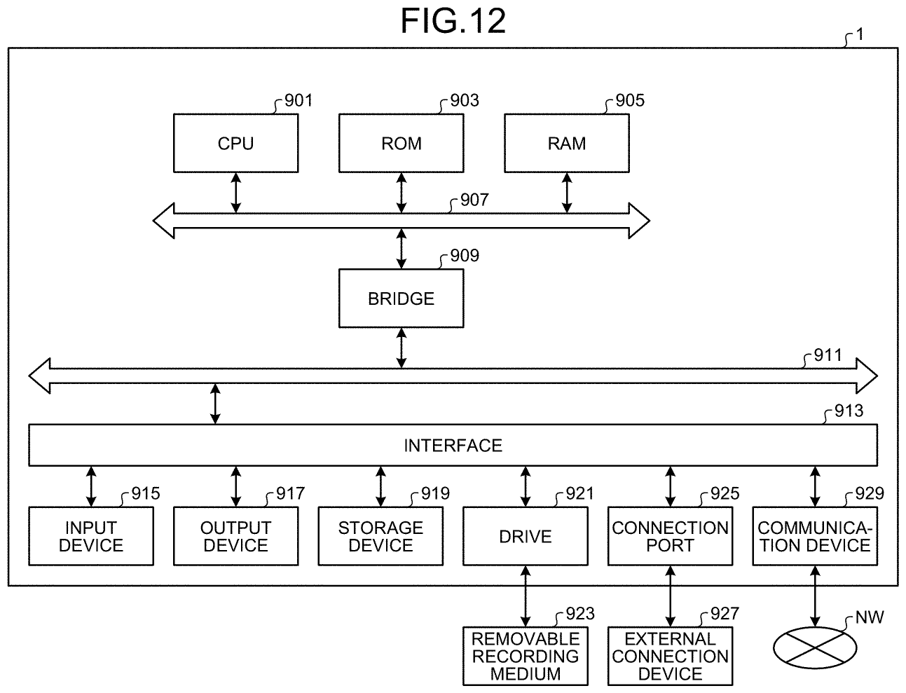

Next, a hardware configuration of the information processing device according to an embodiments of the present disclosure will be described with reference to FIG. 12. FIG. 12 is a block diagram illustrating a hardware configuration example of the information processing device according to an embodiment of the present disclosure. Note that, although a hardware configuration example of the information processing device 1 is illustrated here, a hardware configuration of the information processing device 1a is similar thereto.

As illustrated in FIG. 12, the information processing device 1 includes a central processing unit (CPU) 901, a read only memory (ROM) 903, and a random access memory (RAM) 905. In addition, the information processing device 1 includes a host bus 907, a bridge 909, an external bus 911, an interface 913, an input device 915, an output device 917, a storage device 919, a drive 921, a connection port 925, and a communication device 929. The information processing device 1 may include a processing circuit called a digital signal processor (DSP) or an application specific integrated circuit (ASIC) instead of or in addition to the CPU 901.

The CPU 901 functions as an arithmetic processing device and a control device, and controls the overall operation or a part thereof in the information processing device 1 according to various programs recorded in the ROM 903, the RAM 905, the storage device 919, or a removable recording medium 923. For example, the CPU 901 controls the overall operation of each functional unit included in the information processing device 1 in the above embodiment. The ROM 903 stores a program, an operation parameter, and the like used by the CPU 901. The RAM 905 primarily stores a program used in execution of the CPU 901, a parameter that appropriately changes in the execution, and the like. The CPU 901, the ROM 903, and the RAM 905 are connected to each other by the host bus 907 constituted by an internal bus such as a CPU bus. Furthermore, the host bus 907 is connected to the external bus 911 such as a peripheral component interconnect/interface (PCI) bus via the bridge 909.

The input device 915 is a device operated by a user, such as a mouse, a keyboard, a touch panel, a button, a switch, or a lever. The input device 915 may be, for example, a remote control device using an infrared ray or another radio wave, or an external connection device 927 such as a mobile phone corresponding to operation of the information processing device 1. The input device 915 includes an input control circuit that generates an input signal on the basis of information input by a user and outputs the input signal to the CPU 901. By operating the input device 915, a user inputs various types of data to the information processing device 1 or instructs the information processing device 1 to perform processing operation.

The output device 917 is constituted by a device capable of visually or aurally notifying a user of acquired information. The output device 917 can be, for example, a display device such as an LCD, a PDP, or an OELD, a sound output device such as a speaker or a headphone, or a printer device. The output device 917 outputs a result obtained by processing of the information processing device 1 as a video such as a text or an image, or as a sound such as audio.

The storage device 919 is a data storage device constituted as an example of a storage of the information processing device 1. The storage device 919 is constituted by, for example, a magnetic storage device such as a hard disk drive (HDD), a semiconductor storage device, an optical storage device, or a magneto-optical storage device. The storage device 919 stores a program and various types of data executed by the CPU 901, various types of data acquired from the outside, and the like.

The drive 921 is a reader/writer for the removable recording medium 923 such as a magnetic disk, an optical disk, a magneto-optical disk, or a semiconductor memory, and is built in or externally attached to the information processing device 1. The drive 921 reads information recorded in the attached removable recording medium 923 and outputs the information to the RAM 905. In addition, the drive 921 writes a record in the attached removable recording medium 923.

The connection port 925 is a port for directly connecting a device to the information processing device 1. The connection port 925 can be, for example, a universal serial bus (USB) port, an IEEE 1394 port, or a small computer system interface (SCSI) port. In addition, the connection port 925 may be an RS-232C port, an optical audio terminal, a high-definition multimedia interface (HDMI) (registered trademark) port, or the like. By connecting the external connection device 927 to the connection port 925, various types of data can be exchanged between the information processing device 1 and the external connection device 927.

The communication device 929 is, for example, a communication interface constituted by a communication device or the like for connection to a communication network NW. The communication device 929 can be, for example, a communication card for wired or wireless local area network (LAN), Bluetooth (registered trademark), or wireless USB (WUSB). In addition, the communication device 929 may be a router for optical communication, a router for asymmetric digital subscriber line (ADSL), a modem for various types of communication, or the like. The communication device 929 transmits and receives a signal and the like to and from the Internet or another communication device using a predetermined protocol such as TCP/IP. In addition, the communication network NW connected to the communication device 929 is a network connected in a wired or wireless manner, and is, for example, the Internet, a home LAN, infrared communication, radio wave communication, or satellite communication.

Note that the technical scope of the present disclosure is not limited to the above-described embodiments as they are, and various modifications can be made without departing from the gist of the present disclosure. In addition, components of different embodiments and modifications may be appropriately combined with each other.

For example, in the above embodiments, the information processing system 4 includes the information processing device 1 and the measurement device 3, but the present technology is not limited to such an example. For example, the information processing device 1 may have a function (measurement function) of the measurement device 3. In this case, the information processing system 4 is implemented by the information processing device 1. In addition, the measurement device 3 may have the functions of the information processing device 1. In this case, the information processing system 4 is implemented by the measurement device 3. In addition, the measurement device 3 may have some of the functions of the information processing device 1, and the information processing device 1 may have some of the functions of the measurement device 3.

In addition, the effects of the embodiments described here are merely examples and are not limited, and other effects may be provided.

Note that the present technology can also have the following configurations.
(1)

An information processing device comprising a display control unit that displays a correspondence relationship between a gate obtained by gate analysis of an analysis target having a plurality of attributes based on the plurality of attributes and a cluster obtained by cluster analysis of the analysis target based on the plurality of attributes.
(2)

The information processing device according to (1), further comprising

a calculation unit that calculates a matching degree between the gate and the cluster, wherein

the display control unit displays the gate such that the gate corresponds to the cluster on a basis of a matching degree calculated by the calculation unit.
(3)

The information processing device according to (2), wherein

the analysis target is a plurality of cells, and

the calculation unit calculates the matching degree using a confusion matrix based on the number of cells included in the gate and the number of cells included in the cluster.
(4)

The information processing device according to (3), wherein the plurality of attributes is intensities corresponding to a plurality of fluorescent dyes detected from the plurality of cells labeled with the plurality of fluorescent dyes.
(5)

The information processing device according to (2), (3) or (4), further comprising

a reception unit that receives selection of a cluster, wherein

the calculation unit calculates a matching degree with a cluster received by the reception unit for all the gates, and

the display control unit displays a gate having a highest matching degree with the cluster received by the reception unit by a display method different from display methods of the other gates.
(6)

The information processing device according to (5), wherein the different display method is a different color.
(7)

The information processing device according to (5) or (6), wherein the display control unit displays a parent gate of the gate having the highest matching degree in a color different from a color of the gate having the highest matching degree.
(8)

The information processing device according to (2), (3) or (4) wherein

the calculation unit calculates a matching degree for combinations of all the clusters and all the gates, and

the display control unit displays each of the gates by using a display color of a cluster having a highest matching degree.
(9)

A display method comprising

a processor displaying a correspondence relationship between a gate obtained by gate analysis of an analysis target having a plurality of attributes based on the plurality of attributes and a cluster obtained by cluster analysis of the analysis target based on the plurality of attributes.
(10)

A program for causing a computer to function as

a display control unit that displays a correspondence relationship between a gate obtained by gate analysis of an analysis target having a plurality of attributes based on the plurality of attributes and a cluster obtained by cluster analysis of the analysis target based on the plurality of attributes.
(11)

An information processing system comprising:

a measurement device including a measurement unit that irradiates a measurement target with light, detects fluorescence emitted from the measurement target, and measures a fluorescence intensity; and

an information processing device including a display control unit that displays a correspondence relationship between a gate obtained by gate analysis based on a plurality of fluorescence intensities measured by the measurement device and a cluster obtained by cluster analysis based on the plurality of fluorescence intensities.
(12)

The information processing system according to (11), wherein the measurement device is a flow cytometer.

### REFERENCE SIGNS LIST

1, 1a INFORMATION PROCESSING DEVICE

MEASUREMENT DATA

MEASUREMENT DEVICE

4, 4a INFORMATION PROCESSING SYSTEM

GATE PROCESSING UNIT

GATE INFORMATION STORAGE UNIT

CLUSTERING PROCESSING UNIT

CLUSTERING DATA STORAGE UNIT

CLUSTER SELECTION UNIT

16, 16a MATCHING DEGREE CALCULATION UNIT

17, 17a MATCHING INFORMATION OUTPUT UNIT

1. An information processing device comprising a display control unit that displays a correspondence relationship between a gate obtained by gate analysis of an analysis target having a plurality of attributes based on the plurality of attributes and a cluster obtained by cluster analysis of the analysis target based on the plurality of attributes.

2. The information processing device according to claim 1, further comprising
a calculation unit that calculates a matching degree between the gate and the cluster, wherein
the display control unit displays the gate such that the gate corresponds to the cluster on a basis of a matching degree calculated by the calculation unit.

3. The information processing device according to claim 2, wherein
the analysis target is a plurality of cells, and
the calculation unit calculates the matching degree using a confusion matrix based on the number of cells included in the gate and the number of cells included in the cluster.

4. The information processing device according to claim 3, wherein the plurality of attributes is intensities corresponding to a plurality of fluorescent dyes detected from the plurality of cells labeled with the plurality of fluorescent dyes.

5. The information processing device according to claim 2, further comprising
a reception unit that receives selection of a cluster, wherein
the calculation unit calculates a matching degree with a cluster received by the reception unit for all the gates, and
the display control unit displays a gate having a highest matching degree with the cluster received by the reception unit by a display method different from display methods of the other gates.

6. The information processing device according to claim 5, wherein the different display method is a different color.

7. The information processing device according to claim 6, wherein the display control unit displays a parent gate of the gate having the highest matching degree in a color different from a color of the gate having the highest matching degree.

8. The information processing device according to claim 2, wherein
the calculation unit calculates a matching degree for combinations of all the clusters and all the gates, and
the display control unit displays each of the gates by using a display color of a cluster having a highest matching degree.

9. A display method comprising
a processor displaying a correspondence relationship between a gate obtained by gate analysis of an analysis target having a plurality of attributes based on the plurality of attributes and a cluster obtained by cluster analysis of the analysis target based on the plurality of attributes.

10. A program for causing a computer to function as
a display control unit that displays a correspondence relationship between a gate obtained by gate analysis of an analysis target having a plurality of attributes based on the plurality of attributes and a cluster obtained by cluster analysis of the analysis target based on the plurality of attributes.

11. An information processing system comprising:
a measurement device including a measurement unit that irradiates a measurement target with light, detects fluorescence emitted from the measurement target, and measures a fluorescence intensity; and
an information processing device including a display control unit that displays a correspondence relationship between a gate obtained by gate analysis based on a plurality of fluorescence intensities measured by the measurement device and a cluster obtained by cluster analysis based on the plurality of fluorescence intensities.

12. The information processing system according to claim 11, wherein the measurement device is a flow cytometer.
